(19) United States
(12) Patent Application Publication
(10) Pub. No.: US 2016/0280881 A1
(43) Pub. Date: Sep. 29, 2016

(54) A METHOD FOR DEGRADING A PLASTIC

(71) Applicant: CARBIOS, Saint - Beauzire (FR)

(72) Inventors: CÉDRIC BOISART, BELBERAUD (FR); EMMANUEL MAILLE, ENNEZAT (FR); FRÉDÉRIQUE GUILLAMOT, GERZAT (FR)

(21) Appl. No.: 15/034,655

(22) PCT Filed: Nov. 4, 2014
(86) PCT No.: PCT/EP2014/073742

§ 371 Date: May 5, 2016

(30) Foreign Application Priority Data
Nov. 5, 2013 (EP) 13306522.7

(51) Int. Cl. C08J 11/10; B09B 3/00; C12P 7/56

(52) U.S. Cl. CPC C08J 11/105; C12P 7/56; B09B 3/00; C08J 2367/04; C08J 2367/02; C08J 2377/06; C08J 2323/08; C08J 2329/04

(57) ABSTRACT

The present invention relates to a method for degrading a plastic containing non-biodegradable polymers comprising submitting said plastic to at least one enzyme for modifying a polymer of said plastic which has a methane potential less than 5 Nm3/t+/−20%, wherein at least one product resulting from the modification exhibits a methane potential greater than 10 Nm3/t+/−20%.

The present invention relates to a method for degrading a plastic containing non-biodegradable polymers. More particularly, the invention relates to a biological method for modifying at least one non-biodegradable polymer of a plastic, for creating a material (e.g., intermediate products) that exhibits a methane potential. The invention also relates to a biological method for improving the methane potential of a plastic. The invention also concerns a methanization process using such products or materials.

### CONTEXT OF THE INVENTION

Plastics are inexpensive and durable materials, which can be used to manufacture a variety of products that find use in a wide range of applications, so that the production of plastics has increased dramatically over the last decades. Among them, one of the largest group of thermoplastics is the group of polyolefin plastics. As an example, in Europe, polyolefins represent nearly half of the total volume of the produced plastics. The two most important and common polyolefins are polyethylene (PE) and polypropylene (PP), which are very popular due to their low cost and wide range of applications. For instance, polyolefins are widely used in packaging (trays, containers, bottles, bags, etc.), for blown film, as well as under garments for wetsuits. Polyolefins are also used in agricultural industry for crop propagation films used to cover seed or planted seedlings.

About 40% of plastics are used for single-use disposable applications or for short-lived products that are discarded within a year of manufacture. This amount represents around 100 million tons of plastic waste per year, most of them coming from packaging applications. A large part of the polymers involved, such as polyolefins, polyethylene terephthalate (PET), polyvinyl chloride, polystyrene, polyurethane, polycarbonate, polyamides are non-biodegradable polymers. Because these packaging plastics are the major plastics to be dumped in the environment and due to their recalcitrant nature, they persist in the environment and generate increasing environmental problems.

One solution to reduce environmental and economic impacts correlated to the accumulation of these plastics is closed-loop recycling wherein plastic material is mechanically reprocessed to manufacture new products. For example, PET, PE or PP wastes are subjected to successive treatments leading to recycled PET, PE or PP which are collected, sorted, pressed into bales, crushed, washed, chopped into flakes, melted and extruded in pellets and offered for sale. Then, these recycled PET, PE or PP may be used to create textile fibers, plastic tubes for the construction industry or plastic films, plastic sheets, or new packaging such as flasks or blister packs, etc.

However, these plastic recycling processes require an efficient upstream sorting process and use huge amounts of electricity, particularly during the extruding step. The equipment used is also expensive, leading to high prices, which may be non-competitive compared to virgin plastic. Moreover, the recycled plastic loses gradually its interesting properties due to the recycling process, and become less interesting compared to virgin plastics.

A solution to reduce the impact of non-biodegradable polymers spread in the environment would be to make them biodegradable. However, the hydrophobicity, high molecular weight, chemical and structural composition of most of them hinders their biodegradation. Different physical, chemical and biochemical approaches have been developed for enhancing their biodegradation. For instance, the rate of the biodegradation can be enhanced by blending the non-biodegradable polymer with biodegradable natural polymers, such as starch or cellulose, or with synthetic polymers, such as poly lactic acid (PLA) and/or by mixing them with prooxidants. However, many of the corresponding compositions that have enhanced degradability have only limited applications due to their difficult processability, cost and/or final properties.

Thus, a need exists for an upgraded process able to degrade or treat non-biodegradable polymer plastic and to enhance their economical value.

### SUMMARY OF THE INVENTION

The inventors now propose a biological process for modifying non-biodegradable polymers contained in a plastic in order to improve their biodegradability. More particularly, the invention discloses a biological method for improving or creating methane potential of non-biodegradable polymer plastics or essentially non-biodegradable polymers, thereby allowing methane fermentation of at least a part of the resulting products. More particularly, the inventors show that it is possible to use enzymes which are able to cleave chains (e.g. carbon-carbon bonds or esters bonds) in non-biodegradable polymers, to breakdown their crystalline structure and generate high methane potential material. The process of the invention allows the production of material (e.g., intermediate products) from non-biodegradable polymers that exhibit a methane potential. Such material represents a valuable substrate that may be further metabolized (i.e.: methane fermentation process), leading to the formation of methane-containing biogas.

In this regard, it is an object of the invention to provide a method for treating a plastic comprising exposing or contacting the plastic to at least one enzyme for modifying a polymer of said plastic which has a methane potential less than 5 Nm3/t+/−20%, and preferably less than 1 Nm3/t+/−20%, wherein at least one product resulting from the modification exhibits a methane potential greater than 10 Nm3/t+/−20%.

It is a purpose of the invention to alter such a non-biodegradable polymer included in a plastic to obtain an intermediate product exhibiting a higher methane potential than the original polymer. Preferably, the methane potential of at least one intermediate product resulting of the process of the invention is above 30 Nm3/t, more preferably above 300 Nm3/t and more generally comprised between 10 and 1000 Nm3/t.

A further object of the invention relates to a method for improving the methane potential of a non-biodegradable polymer plastic comprising exposing said plastic to an enzyme or a microorganism that cleaves carbon-carbon bonds or esters bonds, thereby breaking down its crystalline structure and so increasing the methane potential. The method preferably increases the methane potential by ten folds at least, and more preferably by 30 fold at least. A further object of the invention relates to a method for transforming a plastic comprising exposing said plastic to an enzyme or a microorganism that cleaves carbon-carbon bonds or esters bonds, thereby breaking down its crystalline structure.

A further object of the invention is a composition comprising an essentially non-biodegradable polymer and an enzyme or a microorganism that cleaves carbon-carbon bonds or esters bonds.

Advantageously, the enzyme is a hydrolase selected from the group consisting of a cutinase, lipase, esterase, carboxylesterase, p-nitrobenzylesterase, protease, serine protease, amidase, aryl-acylamidase, urethanase, oligomer hydrolase such as 6-aminohexanoate cyclic dimer hydrolase, 6-aminohexanoate dimer hydolase or an oxidative enzyme selected from the group consisting of a laccase, lipoxygenase, peroxidase, haloperoxidase, mono-oxygenase, di-oxygenase and hydroxilase.

In a particular embodiment, the enzyme is used together with at least one mediator compound or cofactor mediating the enzymatic modification. Advantageously, a mediator compound or cofactor is used together with an oxidative enzyme.

Preferentially, the non-biodegradable polymer, i.e. with a methane potential less than 5 Nm3/t+/−20%, and more preferably less than 1 Nm3/t+/−20%, is selected from the group consisting of polyolefins, ethylene vinyl alcohol (EVOH), poly lactic acid (PLA), polyethylene terephthalate (PET), polybutylene terephthalate (PBT), polytrimethylene terephthalate (PTT), polyethylene isosorbide terephthalate (PEIT), polyethylene furanoate (PEF), polyamide (PA), polyamide-6 or Poly(ε-caprolactam) or polycaproamide (PA6), polyamide-6,6 or Poly(hexamethylene adipamide) (PA6,6), Poly(l1-aminoundecanoamide) (PA11), polydodecanolactam (PA12), poly(tetramethylene adipamide) (PA4,6), poly(pentamethylene sebacamide) (PA5,10), polyhexamethylene nonanediamideaamide (PA6,9), poly(hexamethylene sebacamide) (PA6,10), poly(hexamethylene dodecanoamide) (PA6,12), poly(m-xylylene adipamide) (PAMXD6), polyhexamethylene adipamide/polyhexamethyleneterephtalamide copolymer (PA66/6T), polyhexamethylene adipamide/polyhexamethyleneisophtalamide copolymer (PA66/6I) polyurethane (PU), polyvinyl chloride (PVC), polystyrene (PS), acrylonitrile butadiene styrene (ABS), poly(oxide phenylene) (PPO), polycarbonate (PC), copolymer of phosphono and carboxylic acid (PCA), high molecular weight polyacrylate, polymethacrylate methyle (PMMA), polyoxymethylene (POM), styrene acrylonitrile (SAN), polyester polymer alloy (PEPA), polyethylene naphthalate (PEN), styrene-butadiene (SB) and blends/mixtures of these materials.

In a particular embodiment, the non-biodegradable polymer is a polyolefin, preferably selected from the group consisting of polyethylene, polypropylene, polymethylpentene, polybutene-1, polyisobutylene, ethylene propylene rubber, ethylene propylene diene monomer rubber.

In another particular embodiment, the non-biodegradable polymer is a polyester polymer, preferably selected from the group consisting of poly lactic acid (PLA), polyethylene terephthalate (PET), polybutylene terephthalate (PBT), polytrimethylene terephthalate (PTT), polyethylene isosorbide terephthalate (PEIT), polyethylene furanoate (PEF).

In a particular embodiment, the non-biodegradable polymer is a polyamide polymer, preferably selected from the group consisting of polyamide-6 or Poly(ε-caprolactam) or polycaproamide (PA6), polyamide-6,6 or Poly(hexamethylene adipamide) (PA6,6), Poly(l1-aminoundecano amide) (PA11).

In a particular embodiment, the plastic further comprises at least one polymer selected from the group consisting of, aliphatic polyester, polyvinyl alcohol, cellulose, polylactic acid (PLA), polyhydroxyalkanoate (PHA), starch-based polymers, poly(butylene adipate-co-terephthalate) (PBAT), polybutylene succinate (PBS), polybutylene succinate adipate (PBSA), and polycaprolactone (PCL).

The plastic may be pretreated prior to the alteration step. More particularly, the pretreatment may include a mechanical and/or physical and/or chemical modification of the plastic, like cutting and impact, crushing and grinding, fractionation, cryogenic cooling step, dessicating, dehydration, agglomeration, or granulation.

In a particular embodiment, the plastic may be further sorted, washed and/or biologically cleaned prior to degradation.

In a particular embodiment, the plastic is contacted with at least one microorganism expressing at least one enzyme able to modify a non-biodegradable polymer, or extract thereof. The microorganism may further produce at least one mediator compound or cofactor mediating the enzymatic non-biodegradable polymer modification. Advantageously, a microbial consortium can be used. A lipophilic and/or a hydrophilic agent may be added to enhance the biological treatment.

For instance, the microorganism may be selected from the group consisting of Amycolatopsis, Tritirachium, Kibdelosporangium, Actinomadura, Bionectria, Thermomonospora, Isaria, Bacillus, Acinetobacter, Arthrobacter, Pseudomonas, Sphingomonas, Saccharomyces, Aspergillus, Fusarium, Beauveria, Brevibacillus, Candida, Chaetomium, Cladosporium, Comamonas, Coriolus, Coryneformes, Corynebacterium, Cunninghamella, Delftia, Dictyoglomus, Diplococcus, Engyodontium, Enterobacter, Flavobacterium, Gliocladium, Hansenula, Kluyveromyces, Leptothrix, Listeria, Microbacterium, Micrococcus, Moraxella, Mortierella, Mucor, Mycobacterium, Nocardia, Paecylomyces, Paenibacillus, Penicillium, Phanerochaete, Pleurotus, Proteobacterium, Proteus, Pullularia, Rahnella, Ralstonia, Rhodococcus, Saccharomyces, Serratia, Sphingomonas, Streptomyces, Staphylococcus, Stenotrophomonas, Streptococcus, Talaromyces, Trametes, Trichoderma, and Vibrio.

According to the invention, the method may further comprise subjecting the product exhibiting a methane potential to a methane fermentation step. The modification step and the methane fermentation step may be performed sequentially or simultaneously, in the presence of other wastes than plastics, such as organic wastes or chemical components.

These and the other objects and embodiments of the invention will become more apparent after the detailed description of the invention, including preferred embodiments thereof given in general terms.

### DETAILED DESCRIPTION OF THE INVENTION

The present invention refers to a biological process for altering or transforming or converting non-biodegradable polymer containing materials, i.e. plastics integrating polymers with a potential methane less than 5 Nm3/t+/−20%, and thereby favoring their further methanization into biogas rich in methane. More particularly, the inventors disclose a novel method to bio-alter non-biodegradable polymers in a way that produces particular molecules, which further allows the production of a renewable fuel or gas, alternative to fossil ones.

### DEFINITIONS

The present disclosure will be best understood by reference to the following definitions.

Within the context of the invention, the term “non-biodegradable polymer plastic” or “non-biodegradable polymer containing material” refers to any item made from at least one plastic material, such as plastic sheet, tube, rod, profile, shape, massive block etc., which contains at least one polymer with a methane potential, preferably measured at a temperature of 40° C. or less, less than 5 Nm3/t+/−20% and preferably less than 1 Nm3/t+/−20%. The plastic may further contain other substances or additives, such as plasticizers, mineral or organic fillers. More preferably, the non-biodegradable polymer plastic is a manufactured product like packaging, agricultural films, disposable items or the like. It can be mixed with other wastes such as organic wastes or chemical components (soap, surfactants etc).

A “non-biodegradable polymer” refers to a chemical compound or mixture of compounds whose structure is constituted of multiple repeating units linked by covalent chemical bonds and that has a methane potential, preferably measured at a temperature of 40° C. or less, less than 5 Nm3/t+/−20%, and preferably less than 1 Nm3/t+/−20%. Within the context of the invention, the expressions “non-biodegradable polymer” and “original polymer” are used interchangeably to refer to such a polymer. In a particular embodiment, the non-biodegradable polymers are selected from the group consisting of polyolefins, ethylene vinyl alcohol (EVOH), poly lactic acid (PLA), polyethylene terephthalate (PET), polybutylene terephthalate (PBT), polytrimethylene terephthalate (PTT), polyethylene isosorbide terephthalate (PEIT), polyethylene furanoate (PEF), polyamide (PA), polyamide-6 or Poly(ε-caprolactam) or polycaproamide (PA6), polyamide-6,6 or Poly(hexamethylene adipamide) (PA6,6), Poly(11-aminoundecanoamide) (PA11), polydodecanolactam (PA12), poly(tetramethylene adipamide) (PA4,6), poly(pentamethylene sebacamide) (PA5,10), polyhexamethylene nonanediamideaamide (PA6,9), poly(hexamethylene sebacamide) (PA6,10), poly(hexamethylene dodecanoamide) (PA6,12), poly(m-xylylene adipamide) (PAMXD6), polyhexamethylene adipamide/polyhexamethyleneterephtalamide copolymer (PA66/6T), polyhexamethylene adipamide/polyhexamethyleneisophtalamide copolymer (PA66/6I) polyurethane (PU), polyvinyl chloride (PVC), polystyrene (PS), acrylonitrile butadiene styrene (ABS), poly(oxide phenylene) (PPO), polycarbonate (PC), copolymer of phosphono and carboxylic acid (PCA), high molecular weight polyacrylate, polymethacrylate methyle (PMMA), polyoxymethylene (POM), styrene acrylonitrile (SAN), polyester polymer alloy (PEPA), polyethylene naphthalate (PEN), styrene-butadiene (SB), and blends/mixtures of these materials.

As used herein, the terms “polyolefin” or “polyolefin polymer” are used interchangeably and refer to olefin polymers, especially ethylene and propylene polymers, and copolymers, and to polymeric materials having at least one olefinic comonomer, such as ethylene vinyl acetate copolymer and ionomer. Polyolefin polymers can be linear, branched, cyclic, aliphatic, aromatic, substituted, or unsubstituted. Included in the term “polyolefin polymers” are homopolymers of olefin, copolymers of olefin, copolymers of an olefin and a non-olefinic comonomer copolymerizable with the olefin, such as vinyl monomers, modified polymers of the foregoing, and the like.

As used herein, the terms “polyester” or “polyester polymer” are used interchangeably and refer to polymers containing an ester functional group in their main chain. Esters are generally derived from a carboxylic acid and an alcohol. Polyester polymers can be aromatic, aliphatic or semi-aromatic. Preferably, the polyester of the invention are synthetic polyesters.

As used herein, the terms “polyamide” or “polyamide polymer” are used interchangeably and refer to polymers which repeating units linked by amide bonds. Polyamide polymers can be aromatic, aliphatic or semi-aromatic

Modified polyolefin polymers, versus raw polyolefin polymers, include modified polymers prepared by copolymerizing the homopolymer of the olefin or copolymer thereof with an unsaturated carboxylic acid, e.g., maleic acid, fumaric acid or the like, or a derivative thereof such as the anhydride, ester metal salt or the like. It could also be obtained by incorporating into the olefin homopolymer or copolymer, an unsaturated carboxylic acid, e.g., maleic acid, fumaric acid or the like, or a derivative thereof such as the anhydride, ester metal salt or the like. Examples of modified polyolefin polymers are oxo-polyolefin polymers.

Within the context of the invention, the term “derived from a microorganism” in relation to an enzyme or (poly)peptide indicates that the enzyme or (poly)peptide has been isolated from such a microorganism, or that the enzyme or (poly)peptide comprises all or a biologically active part of the amino acid sequence of an enzyme or (poly)peptide isolated or characterized from such a microorganism.

As used herein, the “methane potential” or “bio-methane potential (BMP)” of a given material or compound, refers to the methane producing ability of said material or compound, for instance in normal cubic meter (Nm3) of methane per ton of raw material (at 0° C.=273K and 1 atm=101325 Pa). More particularly, the methane potential refers to the amount of organic carbon in the given material that can be anaerobically converted to methane during anaerobic degradation in the presence of anaerobic bacteria under normal conditions of temperature and pressure. The methane potential of a compound may be readily assessed by the one skilled in the art according to known methods (see e.g., Kleerebezem et al. Applied and Environmental Microbiology, 1999, 68, 1152-1160; Esposito et al. The Open Environmental engineering Journal, 2012, 5, 1-8). According to the invention, the methane potential of a given material or compound is preferably measured at a temperature of 40° C. or less.

The present invention proposes to degrade at least one non-biodegradable polymer contained in a plastic, so that at least one intermediate product resulting from this degradation exhibits a methane potential greater than 10 Nm3/t, and preferably greater than 30 Nm3/t. It is a purpose of the invention to convert a non-biodegradable polymer into intermediate products, at least one of which being further methanisable (i.e.: usable in a methane fermentation process for producing methane).

The process of the invention may be used for degrading all kinds of non-biodegradable polymer plastic. Preferably, the non-biodegradable polymer plastic contains at least one polyolefin, and/or polyester and/or polyamide. The process of the invention may be applied on domestic plastic wastes, including plastic bottles, plastic bags, plastic packaging, etc. The process of the invention may further be applied to agricultural plastics, such as polyethylene (PE) film and compost bags, or to plastics used in the automotive industry.

In a particular embodiment, the plastic contains polyolefin polymers mixed with at least one non polyolefin polymer. For instance, the polyolefin plastic further contains aliphatic polyester, polyvinyl alcohol, cellulose, PLA, PHA, starch-based polymers, poly(butylene adipate-co-terephthalate) (PBAT), polybutylene succinate (PBS), polybutylene succinate adipate (PBSA), and/or polycaprolactone.

In a particular embodiment, the plastic contains only polyolefin polymers. More preferably, the polyolefin polymers are raw polymers, versus oxo-polyolefin polymers, which contain for instance prooxidant metal ions.

Preferred polyolefins are for instance, polypropylene, polyethylene such as high density polyethylene (HDPE), low density polyethylene (LDPE) and linear low density polyethylene (LLDPE), mixtures comprising these polymers, for example, mixtures of polypropylene with polyisobutylene, polypropylene with polyethylene (for example PP/HDPE, PP/LDPE) and mixtures of different types of polyethylene (for example LDPE/HDPE), or ethylene or propylene copolymers for example ethylene/propylene, LLDPE and its mixtures with LDPE, propylene/butene-1, ethylene/hexene, ethylene/ethylpentene, ethylene/heptene, ethylene/octene, propylene/isobutylene, ethylene/butane-1, propylene/butadiene, and terpolymers of ethylene with propylene and a diene, such as hexadiene, dicyclopentadiene or ethylidene-norbornene. The polyolefins of the invention may also be cross-linked.

It is generally recognized that polyolefin polymers are bioinert that is they are highly resistant to assimilation by microorganisms such as fungi, bacteria and the like. Furthermore, in commercial polyolefin polymers, having relatively high molecular mass values, there are very few ends of molecules accessible on or near the surfaces of the plastics made from them. It is an object of the present invention to provide enzymes suitable for altering the polymer chain so that said polymer chain may undergo an oxidative degradation and that its molecular mass is reduced. Advantageously, the products resulting of the modification step exhibit a reduced molecular mass value and incorporate polar, oxygen-containing groups such as acid, alcohol and ketone.

According to the invention, such degrading enzymes may be a cutinase, lipase, esterase, carboxylesterase, p-nitrobenzylesterase, protease, serine protease, amidase, aryl-acylamidase, urethanase, oligomer hydrolase, laccase, peroxidase, haloperoxidase, lipoxygenase, mono-oxygenase, di-oxygenase and hydroxilase etc., depending on the polymer to hydrolyze. For instance, the laccase from Rhodococcus ruber DSM 45332 or the commercial laccase from Trametes versicolor can be used to oxidize polyethylene. Alternatively, or in addition, lignin and manganese peroxidase from Streptomyces sp. or Phanerochaete chrysosporium may be used. In another example, a cutinase (like the one from Thermobifida fusca or Thermobifida alba or Fusarium solani pisi) or a lipase (like lipase PS from Burkholderia cepacia) may be used for treating a plastic product containing PET or PTT. In a particular embodiment, a cutinase (like the one from Fusarium solani) or a aryl-acylamidase (like the one from Nocardia farcinica) or an oligomer hydrolase (like 6-aminohexanoate oligomer hydrolase from Arthrobacter sp.) or an amidase (like the one from Beauveria brongniartii) may be used for treating a plastic product containing PA6 or PA6,6. In another particular example, an esterase from Pseudomonas sp. or Chaetomium globosum may be used for treating a plastic product containing polyurethane. In another particular example, an esterase or a lipase from Arthrobacter sp. or Enterobacter sp. may be used for treating a plastic containing polycarbonate. In another particular example, a serine protease from Actinomadura sp. may be used for treating a plastic containing polylactic acid.

In a particular embodiment, the plastic to treat is contacted with the altering enzyme, which may be natural or synthetic.

For example, the enzyme may be produced by recombinant techniques, or it may be isolated or purified from natural sources, when naturally-occurring, or it may be artificially produced. The enzyme may be in soluble form, or on solid phase. In particular, it may be bound to cell membranes or lipid vesicles, or to synthetic supports such as glass, plastic, polymers, filter, membranes, e.g., in the form of beads, columns, plates and the like.

The enzymes are preferably in isolated or purified form. For instance, enzymes of the invention may be expressed, derived, secreted, isolated, or purified from a microorganism. The enzymes may be purified by techniques known per se in the art, and stored under conventional techniques. The enzymes may be further modified to improve e.g., their stability or activity.

In another embodiment, the non-biodegradable polymer plastic is contacted with a microorganism that synthesizes and excretes the altering enzyme. In the context of the invention the enzyme may be excreted in the culture medium or towards the cell membrane of the microorganism wherein said enzyme may be anchored.

In particular embodiments, Amycolatopsis, Tritirachium, Kibdelosporangium, Actinomadura, Bionectria, Thermomonospora, Isaria, Bacillus, Acinetobacter, Arthrobacter, Pseudomonas, Sphingomonas, Saccharomyces, Aspergillus, Fusarium, Beauveria, Brevibacillus, Candida, Chaetomium, Cladosporium, Comamonas, Coriolus, Coryneformes, Corynebacterium, Cunninghamella, Delftia, Dictyoglomus, Diplococcus, Engyodontium, Enterobacter, Flavobacterium, Gliocladium, Hansenula, Kluyveromyces, Leptothrix, Listeria, Microbacterium, Micrococcus, Moraxella, Mortierella, Mucor, Mycobacterium, Nocardia, Paecylomyces, Paenibacillus, Penicillium, Phanerochaete, Pleurotus, Proteobacterium, Proteus, Pullularia, Rahnella, Ralstonia, Rhodococcus, Saccharomyces, Serratia, Sphingomonas, Streptomyces, Staphylococcus, Stenotrophomonas, Streptococcus, Talaromyces, Trametes, Trichoderma, and/or Vibrio may be used. For instance, Bacillus bacteria produce oxidases, such as laccases, suitable for oxidizing polyolefin polymers. According to the invention, several microorganisms and/or purified enzymes and/or synthetic enzymes may be used together or sequentially to degrade different kinds of non-biodegradable polymers contained in a same plastic and/or different kinds of non-biodegradable polymers contained in a same plastic to degrade simultaneously.

Advantageously, the non-biodegradable polymer plastic is contacted with a culture medium containing the microorganisms, glucose or the like as a carbon source, as well as a nitrogen source which may be assimilated by the microorganisms, including an organic nitrogen source (e.g., peptone, meat extract, yeast extract, corn steep liquor) or an inorganic nitrogen source (e.g., ammonium sulfate, ammonium chloride). If necessary, the culture medium may further contain inorganic salts (e.g., sodium ion, potassium ion, calcium ion, magnesium ion, sulfate ion, chlorine ion, phosphate ion). Moreover, the medium may also be supplemented with trace components such as vitamins, oligoelements and amino acids.

In a particular embodiment, the enzymes and/or microorganisms are used together with at least one mediator compound mediating the enzymatic non-biodegradable polymer modification. For instance, the enzymatic reaction of the invention may be advantageously implemented with an initiator (such as hydroperoxide POOH) that leads to the formation of radicals. In a particular embodiment, ABTS is doubled oxidized in order to play a mediator role. This dication is stable and can be easily regenerated. HBT, acetosyringone and TEMPO may also be used as mediators in the process of the invention.

According to an embodiment of the invention, the plastic is oxidized or hydrolyzed and intermediate molecules are produced, part of them exhibiting a methane potential greater than 10 Nm3/t, +/−20%. For instance, with the method of treatment of the invention, the oxidation of a polyolefin contained in a plastic material (LDPE which has an initial methane potential of 0.5 Nm3/t±20%) produces aliphatic alcohols, aliphatic ketones, alcanes (octadecane), alcenes (1-nonadecene), fatty acids (palmitic acid), esters (ergosta-5,22-dien-3-ol acetate), cyclic compounds (azafrine) which all have a methane potential superior to 10 Nm3/t±20%. In another example, the hydrolysis of a PET which has an initial methane potential inferior to 5 Nm3/t±20% according to the method of treatment of the invention leads to the production of terephthalic acid, mono-(2-hydroxyethyl) terephthalate (MHET) and bis-(2-hydroxyethyl) terephthalate (BHET) which all have a methane potential superior to 10 Nm3/t±20%. In another example, the hydrolysis of a PA6,6 which has an initial methane potential inferior to 5 Nm3/t±20% according to the method of treatment of the invention leads to the production of adipic acid, hexamethylenediamine, 6-aminohexanoate dimer which all have a methane potential superior to 10 Nm3/t±20%.

In the context of the invention, the methane potential is measured using a method adapted from Esposito et al., 2012, wherein the compound of interest is inoculated with anaerobic bacteria for a period of 30 to 60 days at 37° C. in static conditions. Biogas is analyzed for CH4 content with a Gas Chromatograph device such as the Shimadzu Gas Chromatograph model GC-8A with a flame ionization detector. Alternatively, the methane potential is measured using a method based on the metabolic measurement of a methanogen microorganism activity. Such method may be for instance implemented using the Envital® kit from Envolure.

Preferably, the methane potential of at least one product resulting from the degradation process of a given plastic according to the invention is higher than 10 Nm3/t, +/−20%. More generally, the methane potential of at least one product resulting from the degradation process of a given original polymer is preferably comprised between 30 and 1000 Nm3/t.

Advantageously, the resulting product exhibits a methane potential at least 10 times higher, more preferably at least 30 times higher than the methane potential of the original polymer.

In a particular embodiment, the plastic may be preliminary treated to physically change its structure, so as to increase the surface of contact between the polymers and the enzymes. For example, the plastic may be transformed to an emulsion or a powder, prior to be subjected to the process of the invention. Alternatively, the plastic may be mechanically grinded, granulated, pelleted, etc. to reduce the shape and size of the material prior to be subjected to the process of the invention. One skilled in the art knows that the greater the specific surface is, the greater will be the methane potential.

The time required for degradation of a plastic containing non-biodegradable polymers may vary depending on the plastic itself (i.e., nature and origin of the plastic, its composition, shape etc.), the type and amount of microorganisms/enzymes used, as well as various process parameters (i.e., temperature, pH, additional agents, etc.). One skilled in the art may easily adapt the process parameters to the plastic and/or altering enzymes.

In a particular embodiment, the modification step is performed under aerobic conditions. Advantageously, the process is implemented under humid conditions, preferably between 30 and 90% humidity.

Advantageously, the process is implemented at a temperature comprised between 20° C. and 80° C., more preferably between 35° C. and 60° C. More generally, the temperature is maintained below an inactivating temperature, which corresponds to the temperature at which the enzyme is inactivated and/or the microorganism does no more synthesize the enzyme.

According to the invention, the added amount of enzyme for the alteration step may be at least 0.05% by weight of plastic, preferably at least 0.1% and more preferably at least 1%. And the added amount is advantageously at more 50% by weight of plastic and more preferably at more 5%.

The pH of the medium may be in the range of 3 to 10, more preferably between 5.5 and 8.

In a particular embodiment, at least an inductor such as gelatin can be added to the medium to improve enzyme production. A surfactant such as Tween can be added to the medium to modify interface energy between the polymer and the enzyme or microorganism and improve degradation efficiency. The surfactant can be produced by the microorganism used to produce the enzyme. An organic substance could be used to swell the polymer and increase its accessibility to the microorganism or enzyme.

In the context of the invention, the methane potential is measured after an incubation time comprised between 30 and 60 days, according to the following test:

The compound of interest is inoculated with anaerobic bacteria (inoculum) and incubated for a period of 30 to 60 days at 37° C. in static conditions in a flask with septum. Each flask is partially filled with inoculum and the compound of interest, according to a ratio equal to 2 between their VS content (volatile solids). Distillated water is added up to the ½ volume of the flask. Inoculum for the assay is acquired from an active anaerobic reactor (sewage sludge from a high solid anaerobic digester operating on pretreated household waste). None adaptation of the inoculum to the compound of interest is realized before the measurement of the methane potential. Change of inoculum induces around 20% variation of BMP. The compound of interest can be grounded, but the particle size remains above 500 μm for the measurement of the methane potential. Biogas production is monitored throughout the test. The amount of gas produced is measured by inserting a needle connected to a digital manometer and measuring the pressure differential between the sealed assay bottle and the ambient atmosphere. Biogas is analyzed for CH₄ content with a Gas Chromatograph device such as the Shimadzu Gas Chromatograph model GC-8A with a flame ionization detector. A control containing only inoculum and water may be used to determine CH4 production resulting from the inoculum alone.

Alternatively the Envital® kit from Envolure can be used to determine BMP in a 96 well microplate, as more particularly disclosed in the examples.

The method of the invention can be performed before the methanization process in a specific reactor, e.g a stabilizing bioreactor (horizontal or vertical) with an incubation time of 3 days. Alternatively, the method of the invention can be performed during the methanization process, in the global reactor or in the hydrolyse bioreactor where hydrolyse, acidogenesis and acetogenesis are realized before the methanogenesis step.

Further aspects and advantages of the invention will be disclosed in the following examples, which should be considered as illustrative and do not limit the scope of this application.

### EXAMPLES

The examples below illustrate the use of the method for treating plastics of the invention on different kinds of non-biodegradable polymer plastics, in order to improve their methane potential.

### Example 1

### PLA Treatment

Plastic product made of PLA can be methanized at low temperature thanks to the method of the invention. Example 1 shows the treatment of PLA with a serine protease that leads to the production of lactic acid which has a greater methane potential than PLA.

### Plastic Product and Pre-Treatment

Pellets of PLLA were purchased from NaturePlast (ART00120-PLLA001) and were ground by using a cutting mill SM-2000 (Retsch) during 5 min and then sieved with a siever AS 200 (Retsch) during 10 min with an amplitude of 1.5 mm to obtain a powder of 500 μm.

### Protease Production

Actinomadura keratinilytica DSMZ 45195 was obtained from the German Resource Centre for Biological Material (DSMZ, Germany). The strain was maintained on medium 65 recommended by DSMZ.

Batch experiment was performed in a 10-L fermentor (Sartorius® Biostat Cplus). 500 mL of Yeast Malt Broth (YM, Sigma-Aldrich) pre-culture were used to inoculate 4.5 L of basal medium (gelatin 2.4 g/L; (NH₄)₂SO₄ 4 g/L; MgSO₄. 7H₂O 0.2 g/L; yeast extract 0.5 g/L; K₂HPO₄ 4 g/L; KH₂PO₄ 2 g/L adjusted at pH 6.8 with NaOH). The temperature was regulated at 46° C. and the pH maintained at 6.8 with the addition of a 10% (v/v) H₃PO₄ solution. The stirring rate was fixed at 70 rpm to enable a gentle mixing and the aeration rate (0.6 to 1.6 vvm) was regulated to provide the reactor with a dissolved oxygen level higher than 20% of air saturation, in order to avoid any oxygen limitation in the culture. The fermentor was connected to a computer and the MFCS/DA software carried out the on-line acquisition of the controlled parameters (pH, temperature, partial pressure of dissolved oxygen and H₃PO₄ addition) and allowed the monitoring and the regulation of these parameters on-line.

The culture duration was 50 hours. Supernatant, containing the extracellular enzyme, was recovered by centrifugation (13000 g-10 min) and concentrated 40 fold using Amicon Cell 500 mL (Merck Millipore) and a cellulose regenerated membrane with a pore size of 10 KDa (GE Healthcare Life Science). The resulting solution was dialyzed against 50 mM glycine-NaOH pH 10 buffer.

An AKTA Purifier apparatus (GE Healthcare Life Science) was used to carry out polypeptide purification, using an anion exchange purification HiTrap Q FF 1 mL column (GE Healthcare Life Science) with 50 mM glycine-NaOH pH 10 as loading buffer. Elution was carried out with a 0 to 1M NaCl gradient in 50 mM glycine-NaOH pH 10 buffer. The flow-through fraction was used to treat PLA.

### Enzymatic Treatment of Plastic Product

1 g plastic product was incubated with 500 μg of serine protease in 20 mL buffer Tris/HCl 100 mM, pH 8.5 for 48 h at 45° C. with 150 rpm shaking in a 10 kDa dialysis tube (cellulose membrane, width 25 mm, Sigma-Aldrich D9777-100FT).

### Lactic Acid (LA) Assay

After enzymatic treatment, sample was centrifuged (Hettich MIKRO 200 R, Tuttlingen, Germany) at 16,000 g at 0° C. for 15 min. 500 μL of supernatant was brought to an HPLC vial. The HPLC used was a DIONEX P-580 PUMP (Dionex Cooperation, Sunnyvale, USA), with an ASI-100 automated sample injector and a PDA-100 photodiode array detector. For analysis of LA, a column Aminex HPX-87H (300 mm×7.8 mm) was used. Analysis was carried out at 50° C. with a mobile phase. The flow rate was set to 0.5 mL/min and the column was maintained at a temperature of 25° C. The injection volume was 20 μL. Detection of LA was performed with an UV detector. Standards of lactic acid (Sigma-Aldrich L1750-10G) were used for external calibration.

### Methane Potential Analysis

The Envital® kit analysis consists of the use of a bioreactive which uses the reducing potential of cells to convert resazurine into fluorescent molecule. Viable cells in the presence of organic matters convert in a continue way the resazurine into resorufine. The emitted fluorescence (Exc:560 nm-Em:600 nm) is, from then on, proportional in the quantity of degraded organic matter. The used inoculum consisted of mud of digesteur. This mud was filtered in 1.2 μm and successively diluted in an adequate concentration for the implementation of the biological analysis. 50 μL of reactive A (buffer solution pH 7), 100 μL of reactive B (Bioreactive), 100 μL of sample (consisting on the mixture of plastic and enzyme) pre-diluted to 1:10 in water and 30 μL of inoculum were added in each well. The wells were then closed by means of paraffin wax to obtain anaerobic conditions. The microplate was then placed in a microplate reader (Clariostar BMG Labtech) at 35° C. with a measurement every 30 min.

### Results

The treatment of PLA with serine protease allows to obtain 90% hydrolysis, leading to a mixture comprising 80 mg PLA and 920 mg LA. This mixture presented a methane potential of 100 Nm3/t, whereas the methane potential of the original PLA was less than 5 Nm3/t at 35° C. There is thus an interest to treat PLA plastic products by enzymatic hydrolysis to recover LA which has a higher methane potential than PLA.

### Example 2

### Aromatic Polyester Treatment

Plastic product based on aromatic polyester such as PET can be methanized thanks to the method of the invention. Example 2 shows the treatment of PET with a cutinase that leads to the production of terephtalic acid and monoethylene glycol exhibiting a methane potential greater than 5 Nm3/t, contrary to PET.

### Plastic Product and Pre-Treatment

Pellets of PET were purchased from NaturePlast (ART0116-PTI001) and were grounded by using a cutting mill SM-2000 (Retsch) during 5 min and then sieved with a siever AS 200 (Retsch) during 10 min with an amplitude of 1.5 mm to obtain a powder of 500 μm.

### Cutinase Production

Thermobifida cellulosilytica DSM44535 was obtained from the German Resource Centre for Biological Material (DSMZ, Germany). The strain was maintained on LB agar plates and cultivated in 500 mL shaking flasks (200 mL LB medium) at 37° C. and 160 rpm for 24 h. Cells were harvested by centrifugation at 3,200 g and 4° C. for 20 min.

Vector pET26b(+) (Novagen, Germany) was used for expression of cutinase THC_Cut1 from Thermobifida cellulosytica in Escherichia coli BL21-Gold (DE3) (Stratagene, Germany).

The gene Thc_cut1 coding for cutinase was amplified from the genomic DNA of T. cellulosilytica DSM44535 by standard polymerase chain reaction (PCR). On the basis of the known sequence of genes coding for cutinases from T. fusca YX (Genbank accession numbers YP_288944 and YP_288943,33) two primers were designed, 5′-CCCCCGCTCATATGGCCAACCCCTACGAGCG-3′ (forward primer, SEQ ID No 1) and 5′-GTGTTCTAAGCTTCAGTGGTGGTGGTGGTGGTGCTCGAGTGCCAGGCACTGAGAG TAGT-3′ (reverse primer, SEQ ID No 2), allowing amplification of the respective genes without signal peptide and introduction of the 6×His-Tag at the C-terminus of the cutinase. The designed primers included restriction sites NdeI and HindIII for cloning the gene into the vector pET26b( [symbol] )). The PCR was done in a volume of 50 μL with genomic DNA as template, 0.4 μM of each primer, 0.2 mM dNTP's, 5 units Phusion DNA polymerase (Finnzymes) and 1× reaction buffer provided by the supplier. The PCR was performed in a Gene Amp PCR 2200 thermocycler (Applied Biosystems, USA). 35 cycles were done, each cycle with sequential exposure of the reaction mixture to 98° C. (30 s, denaturation), 63° C. (30 s, annealing), and 72° C. (30 s, extension). Plasmids and DNA fragments were purified by Qiagen DNA purification kits (Qiagen, Germany). The purified amplified PCR-products thus obtained were digested with restriction endonucleases NdeI and HindIII (New England Biolabs, USA), dephosphorylated with alkaline phosphatase (Roche, Germany) and ligated to pET26b( [symbol] )) with T4 DNA-ligase (Fermentas, Germany) and transformed in E. coli BL21-Gold(DE3) in accordance to the manufacturer's instructions.

The sequence of the gene was determined by DNA sequencing using the primers 5′-GAGCGGATAACAATTCCCCTCTAGAA-3′ (SEQ ID No 3) and 5′-CAGCTTCCTTTCGGGCTTTGT-3′ (SEQ ID No 4). DNA was sequenced as custom service (Agowa, Germany). Analysis and handling of DNA sequences was performed with Vector NTi Suite 10 (Invitrogen, USA). Sequences of proteins were aligned using the Clustal W program (Swiss EMBnet node server). The nucleotide sequence of the isolated gene has been deposited in the GenBank database under accession number HQ147786 (Thc_cut2).

Freshly transformed E. coli BL21-Gold (DE3) cells were used to inoculate 20 mL of LB-medium supplemented with 40 μg/mL kanamycin and cultivated overnight at 37° C. and 160 rpm. The overnight culture was used to inoculate 200 mL of LB-medium with 40 μg/mL kanamycin to OD600=0.1 and incubated until an OD600=0.6-0.8 was reached. Afterwards the culture was cooled to 20° C. and induced with IPTG at a final concentration of 0.05 mM. Induction was done for 20 h at 20° C. and 160 rpm. The cells were harvested by centrifugation (20 min, 4° C., 3,200 g).

Cell pellet from 200 mL cell culture was resuspended in 30 mL binding buffer (20 mM NaH2PO4*2H2O, 500 mM NaCl, 10 mM imidazole, pH 7.4). The resuspended cells were sonicated with three-times 30-s pulses under ice cooling (Vibra Cell, Sonics Materials, Meryin/Satigny, Switzerland). The lysates were centrifuged (30 min, 4° C., 4,000 g) and filtered through a 0.2 μm membrane. The cell lysate was purified using an Äkta purification system with HisTrap FF columns (elution buffer 20 mM NaH2PO4*2H2O, 500 mM NaCl, 500 mM imidazole, pH 7.4). For characterization of cutinase the HisTag elution buffer was exchanged with 100 mM Tris HCl pH 7.0 by the use of PD-10 desalting columns (GE Healthcare).

Protein concentrations were determined by the Bio-Rad protein assay kit (Bio-Rad Laboratories GmbH) and bovine serum albumin as protein standard. SDS-PAGE was performed corresponding to Laemmli (Laemmli, U. K. Nature 1970, 227 (5259), 680-685) and proteins were stained with Coomassie Brilliant Blue R-250.

All chemicals were of analytical grade from Sigma (Germany).

### Enzymatic Treatment of Plastic Product

1 g plastic product was incubated with 5 μM cutinase in 1 L buffer Tris/HCl 100 mM, pH 7.0 for 7 days at 60° C. with 300 rpm shaking.

### Terephtalic Acid (TA) Assay

After enzymatic treatment, proteins were precipitated using 1:1 (v/v) absolute methanol (Merck) on ice. Samples were centrifuged (Hettich MIKRO 200 R, Tuttlingen, Germany) at 16,000 g at 0° C. for 15 min. 500 μL of supernatant was brought to an HPLC vial and acidified by adding 3.5 μL of 6N HCl. The HPLC used was a DIONEX P-580 PUMP (Dionex Cooperation, Sunnyvale, USA), with an ASI-100 automated sample injector and a PDA-100 photodiode array detector. For analysis of TA, a reversed phase column RP-C18 (Discovery HS-C18, 5 μm, 150×4.6 mm with precolumn, Supelco, Bellefonte, USA) was used. Analysis was carried out with 60% water, 10% 0.01N H2SO4 and 30% methanol as eluent, gradual (15 min) to 50% methanol and 10% acid, gradual (to 20 min) 90% methanol and acid, staying 2 min and then gradual to starting position, 5 min post run. The flow rate was set to 1 mL/min and the column was maintained at a temperature of 25° C. The injection volume was 10 μL. Detection of TA was performed with a photodiode array detector at the wavelength of 241 nm.

### Monoethylene Glycol (MEG) Assay

After enzymatic treatment, proteins were precipitated according to the Carrez-precipitation for detection of MEG. Therefore the samples were brought to a pH of 4 to 6 by adding 6N HCl. 2% of solution Cl (5.325 g K4[Fe(CN)6].3H₂O in 50 mL milliQ water) were added to the samples with a dispenser, after vortexing and incubation for 1 minute, 2% of solution C2 (14.400 g ZnSO4.7H₂O in 50 mL milliQ water) were added. After vortexing and incubation for 5 minutes the samples were centrifuged (30 min, 12600 rpm, 25° C.). The supernatants were filtered through a 0.45 μm filter membrane directly into glass vials for HPLC-RI analyses (Hewlett Packard Series 1100, Detector: Agilent Series 1100). The column ION-300 (Transgenomic, Inc.) was used, the flow was set to 0.1 mL/min and 0.01N H2SO4 was used as a mobile phase. The temperature was set to 45° C. and the injection volume was 40 μl.

### Methane Potential Analysis

The Envital® kit analysis was used in the same way than in Example 1.

### Results

The treatment of PET with cutinase leads to 10% hydrolysis (I.e.: 900 mg PET, 87 mg TA and 32 mg MEG). The resulting mixture exhibited a methane potential of 15 Nm3/t, whereas the PET methane potential was under the limit of quantification (i.e.: less than 5 Nm3/t). The methane potential of a synthetic mixture consisting of 100 mg PET, 779 mg TA and 291 mg MEG has also been evaluated to 20 Nm3/t. In addition, the methane potential of pure MEG has been evaluated to 100 Nm3/t. There is thus an interest to treat PET plastic products by enzymatic hydrolysis to recover MEG which has a higher methane potential than PET.

### Example 3

### Polyamide Treatment

Plastic product based on polyamide such as PA6,6 can be methanized thanks to the invention. Example 2 shows the treatment of PA6,6 with an aryl-acyl amidase of Nocardia farcinica that leads to the production of adipic acid and hexamethylene diamine, which have a methane potential higher than PA6,6.

### Plastic Product and Pre-Treatment

Pellets of PA6,6 were purchased from DuPont (Zytel 101 NC010) and were ground by using a cutting mill SM-2000 (Retsch) during 5 min and then sieved with a siever AS 200 (Retsch) during 10 min with an amplitude of 1.5 mm to obtain a powder of 500 μm.

### Aryl-Acyl-Amidase Production

The aryl-acyl-amidase from Nocardia farcinica described in Heumann et al. (2008) was heterologously expressed in E. coli.

### Enzymatic Treatment of Plastic Product

1 g plastic product was incubated with 5 μM aryl-acyl-amidase in 1 L buffer Tris/HCl 100 mM, pH 7.0 for 7 days at 50° C. with 300 rpm shaking.

### Adipic Acid Assay

See the analysis method (MEG assay) of Example 2.

### Methane Potential Analysis

The Envital® kit analysis was used in the same way than in Example 1.

### Results

The treatment of PA with aryl-acyl-amidase leads to 10% hydrolysis, allowing in the present case to the production of 65 mg adipic acid. The resulting mixture exhibited a methane potential of 15 Nm3/t, whereas the PA methane potential was under the limit of quantification (i.e.: less than 5 Nm3/t). It is interesting to note that the enzymatic treatment may be improved to increase the methane potential. Indeed, methane potential of pure adipic acid and hexamethylene diamine have been evaluated to 45 Nm3/t and 13 Nm3/t, respectively. There is thus an interest to treat PA plastic products by enzymatic hydrolysis to recover its monomers which have a higher methane potential than PA.

### Example 4

### Polyolefin Treatment

Fragments coming from an oxo-degradable polyolefin based on Mn—Fe pro-oxidants were treated by 500 nkat laccase from Trametes versicolor (Sigma) and 0.2 mM 1-hydroxybenzotriazole (HBT) in 50 mM malonate buffer (pH 4.5) at 30° C.

The Envital® kit analysis was used in the same way than in Example 1 and a methane potential of 11 Nm3/t was measured after enzymatic treatment. Methane potential of PE and EVOH, which are both partially degradable, were both less than 5 Nm3/t.

1-17. (canceled)

18. A method for treating a plastic comprising exposing said plastic to at least one enzyme for modifying a polymer of said plastic which has a methane potential less than 5 Nm3/t+/−20%, wherein at least one product resulting from the modification exhibits a methane potential greater than 10 Nm3/t+/−20%.

19. The method according to claim 18, wherein the enzyme is a hydrolase selected from the group consisting of a cutinase, lipase, esterase, carboxylesterase, p-nitrobenzylesterase, protease, serine protease, amidase, aryl-acylamidase, urethanase, oligomer hydrolase or an oxidative enzyme selected from the group consisting of a laccase, lipoxygenase, peroxidase, haloperoxidase, mono-oxygenase, di-oxygenase and hydroxilase.

20. The method according to claim 18, wherein the resulting product exhibits a methane potential comprised between 10 and 1000 Nm3/t.

21. The method according to claim 18, wherein the resulting product exhibits a methane potential at least 10 times higher than the methane potential of the original polymer.

22. The method according to claim 18, wherein the original polymer is selected from the group consisting of polyolefins, ethylene vinyl alcohol (EVOH), poly lactic acid (PLA), polyethylene terephthalate (PET), polybutylene terephthalate (PBT), polytrimethylene terephthalate (PTT), polyethylene isosorbide terephthalate (PEIT), polyethylene furanoate (PEF), polyamide (PA), polyamide-6 or Poly(ε-caprolactam) or polycaproamide (PA6), polyamide-6,6 or Poly(hexamethylene adipamide) (PA6,6), Poly(l1-aminoundecanoamide) (PA11). polydodecanolactam (PA12), poly(tetramethylene adipamide) (PA4,6), poly(pentamethylene sebacamide) (PA5,10), polyhexamethylene nonanediamideaamide (PA6,9), poly(hexamethylene sebacamide) (PA6,10), poly(hexamethylene dodecanoamide) (PA6,12), poly(m-xylylene adipamide) (PAMXD6), polyhexamethylene adipamide/polyhexamethyleneterephtalamide copolymer (PA66/6T), polyhexamethylene adipamide/polyhexamethyleneisophtalamide copolymer (PA66/6I) polyurethane (PL), polyvinyl chloride (PVC), polystyrene (PS), acrylonitrile butadiene styrene (ABS), poly(oxide phenylene) (PPO), polycarbonate (PC), copolymer of phosphono and carboxylic acid (PCA), polyacrylate, polymethacrylate methyle (PMMA), polyoxymethylene (POM), styrene acrylonitrile (SAN), polyester polymer alloy (PEPA), polyethylene naphthalate (PEN), styrene-butadiene (SB), and blends/mixtures of these materials.

23. The method according to claim 18, wherein the original polymer is a polyolefin polymer.

24. The method according to claim 23, wherein the polyolefin polymer is selected from the group consisting of polyethylene, polypropylene, polymethylpentene, polybutene-1, polyisobutylene, ethylene propylene rubber and ethylene propylene diene monomer rubber.

25. The method according to claim 18, wherein the original polymer is a polyester polymer.

26. The method according to claim 25, wherein the polyester polymer is selected from the group consisting of poly lactic acid (PLA), polyethylene terephthalate (PET), polybutylene terephthalate (PBT), polytrimethylene terephthalate (PTT), polyethylene isosorbide terephthalate (PEIT) and polyethylene furanoate (PEF).

27. The method according to claim 18, wherein the original polymer is a polyamide polymer.

28. The method according to claim 27, wherein the polyamide polymer is selected from the group consisting of polyamide-6 or Poly(ε-caprolactam) or polycaproamide (PA6), polyamide-6,6 or Poly(hexamethylene adipamide) (PA6,6) and Poly(11-aminoundecanoamide) (PA11).

29. The method according to claim 18, wherein the plastic further comprises at least one polymer selected from the group consisting of aliphatic polyester, polyvinyl alcohol, cellulose, polylactic acid (PLA), polyhydroxyalkanoate (PHA), starch-based polymers, poly(butylene adipate-co-terephthalate) (PBAT), polybutylene succinate (PBS), polybutylene succinate adipate (PBSA), and polycaprolactone (PCL).

30. The method according to claim 18, comprising a preliminary step for mechanically and/or physically and/or chemically degrading said plastic.

31. The method according to claim 18, wherein the enzyme is used together with at least one mediator compound or cofactor mediating the enzymatic modification of the original polymer.

32. The method according to claim 18, comprising contacting the plastic with at least one microorganism expressing at least one enzyme modifying the original polymer, or extract thereof.

33. The method according to claim 32, wherein the microorganism further produces at least one mediator compound mediating the enzymatic modification of the original polymer.

34. The method according to claim 32, wherein the microorganism is selected from the group consisting of Amycolatopsis, Tritirachium, Kibdelosporangium, Actinomadura, Bionectria, Thermomonospora, Isaria, Bacillus, Acinetobacter, Arthrobacter, Pseudomonas, Sphingomonas, Saccharomyces, Aspergillus, Fusarium, Beauveria, Brevibacillus, Candida, Chaetomium, Cladosporium, Comamonas, Coriolus, Coryneformes, Corynebacterium, Cunninghamella, Delftia, Dictyoglomus, Diplococcus, Engyodontium, Enterobacter, Flavobacterium, Gliocladium, Hansenula, Kluyveromyces, Leptothrix, Listeria, Microbacterium, Micrococcus, Moraxella, Mortierella, Mucor, Mycobacterium, Nocardia, Paecylomyces, Paenibacillus, Penicillium, Phanerochaete, Pleurotus, Proteobacterium, Proteus, Pullularia, Rahnella, Ralstonia, Rhodococcus, Saccharomyces, Serratia, Sphingomonas, Streptomyces, Staphylococcus, Stenotrophomonas, Streptococcus, Talaromyces, Trametes, Trichoderma, and Vibrio.

35. The method according to claim 18, further comprising subjecting the product exhibiting a methane potential to a methane fermentation step.

36. The method according to claim 33, wherein the modification step and the methane fermentation step are performed sequentially or simultaneously.

37. A method for improving the methane potential of a non-biodegradable polymer plastic comprising exposing said plastic to an enzyme or microorganism that cleaves carbon-carbon or esters bonds, wherein the resulting product exhibits a methane potential at least 10 times higher.


SEQUENCE LISTING (4 sequences)

SEQ ID NO: 1 - DNA, 31 bp, artificial sequence
  forward primer
cccccgctcatatggccaacccctacgagcg

SEQ ID NO: 2 - DNA, 59 bp, artificial sequence
  reverse primer
gtgttctaagcttcagtggtggtggtggtggtgctcgagtgccaggcactgagagtagt

SEQ ID NO: 3 - DNA, 26 bp, artificial sequence
  primer
gagcggataacaattcccctctagaa

SEQ ID NO: 4 - DNA, 21 bp, artificial sequence
  primer
cagcttcctttcgggctttgt